(19) United States
(12) Patent Application Publication
(10) Pub. No.: US 2021/0186787 A1
(43) Pub. Date: Jun. 24, 2021

(54) Urine collection device

(71) Applicant: Precy Ordinanza, San Diego, CA (US)

(72) Inventor: Precy Ordinanza, San Diego, CA (US)

(21) Appl. No.: 17/183,765

(22) Filed: Feb. 24, 2021

Related U.S. Application Data
(62) Division of application No. 29/710,846, filed on Oct. 25, 2019.
Application No. 29/710,846 is a division of application No. 16/664,790, filed on Oct. 25, 2019, now abandoned.
(60) Provisional application No. 62/750,344, filed on Oct. 25, 2018.

(51) Int. Cl. A61G 9/00

(52) U.S. Cl. CPC A61G 9/003

(57) ABSTRACT

The present disclosure is related to devices for controlling the flow of urine primarily for bedridden patients while in a hospital setting. A device for protecting a patient from constant exposure to urine which may lead to discomfort, skin irritation, infection and subsequent death. The device is designed to secure the genitalia in a leak proof chamber and draw urine away from the skin.

### PRIORITY STATUS

The present application claims priority to Provisional Patent Application 62/750,344 filed on Oct. 25, 2018. All features and descriptions of the priority application are claimed as disclosed herein as if fully set forth.

### FIELD OF THE INVENTION

The present invention relates to a urine control device. More particularly, but not by way of limitation, the present invention relates to a wearable collection device for collecting urine wherein a portion of the external genitalia resides primarily within the device and allows for urine to be collected and removed without leaking onto the body of the patient particularly when bedridden.

### BACKGROUND OF THE INVENTION

The need to remove body fluids from a person's body starts at birth with diapers and constant care. As one ages or is incapacitated it is not unusual for persons to be in non-mobile situations or mentally incapable of taking care of the excretion of body fluids in a hygienic manner and thus require hygienic assistance. Hygienic assistance is a need particularly in immobile patients because exposure to urine and other excretory fluids can result in significant health effects including bedsores, infections which may lead to debilitating health effects, and possibly death. Currently the most commonly used hygienic assistance devices are bedpans, diapers and internal and external catheters. The limitation with bedpans is that they are only effective when the patient is cognizant of their need to void and can get the attention of staff in time. But there are a large number of cases where the patient is unaware or unable to proactively manage their fluids and require greater assistance. Diapers are a cheap and common way of solving the problem but diapers need to be monitored and changed constantly or they lack the effectiveness of removing urine from the skin and thus can lead to the negative health effects described previously. Diaper rash and skin irritation is a constant source of concern, and some hospitals have gone away from diapers altogether because the diapers can hide and exacerbate skin infections. Diapers thus, may lead to more complications for the patient and more liability for the caretakers. Internal catheters are implanted within the patient and although they provide full protection from urine generally, they are very invasive, painful and uncomfortable so much so that in mentally compromised patients they are often yanked out which may lead to significantly more pain and complications. External catheters such as condom catheters provide an alleviation from the pain and intrusion associated with internal catheters, but they are limited in effectiveness because they are hard to position and place on a patient and easily fall off which requires them to be glued in place, thus leading to some of the painful intrusiveness of the internal catheters. Additionally, there is a very large variability in the size of male genitalia and thus when using the external condom catheters the health personnel are required to have an inventory of variable sizes and must fit the patient according with the right size catheter. Because of the limitations of the present solutions and the great need to protect the ever growing older and bedridden population from significant health effects, the present invention was designed to create a better wearable urine collection device primarily, but not limited to male genitalia.

### SUMMARY OF THE INVENTION

The present invention relies on a device allowing for the insertion of all or a portion of the male external genitalia into an enclosed device wherein the device is able to draw urine away from a users body and into the device chamber and optionally an accompanying storage bag when a collection tube apparatus is present.

The embodiments of the present invention include a bottom base unit piece with a large opening on the bottom side for placement of an elastic flexible membrane which includes a smaller hole. The smaller hole in the membrane is used to secure around a patients genitalia. The bottom piece also includes an optional urine collection tube which may also lead to a secondary collection reservoir and drawn through utilizing negative pressure from a pump, or vacuum source. The bottom piece may optionally further include strap support fixtures and a mating lip for securing a leak proof mate with a top cover piece.

The embodied top cover piece may be see through or not and includes a mating ridge for mating with the bottom piece in a snap type leak proof manner. The top cover piece may optionally have a vent system (preferably a one-way vent system) which allows for a negative pressure apparatus to draw the fluid collected within the device into a secondary reservoir.

When in use an embodied urine control device is attached to the user wherein first the bottom side of the bottom piece is secured against the users body by pulling the shaft of the penis through the membrane opening, and allowing the membrane opening to contract in a leak proof manner around the base of the shaft of the penis. An o-ring edge at the opening of the membrane similar to the rolled ring at the opening end of a traditional condom helps secure a liquid proof seal around the shaft of the penis. Additional elastic or ties may accompany the membrane opening to ensure a leak proof seal.

Once the bottom piece is secure in a leak proof manner the top piece is snapped on to the bottom piece to create a chamber for the genitalia to reside in a leak proof manner. Optionally, the device is further secured to the patient through the use of straps.

Further objects, features, and advantages of the present invention will be apparent to those skilled in the art upon examining the accompanying drawings and upon reading the following description of the preferred embodiments.

### BRIEF DESCRIPTION OF THE DRAWINGS

FIG. 1 depicts an unassembled embodied urine control device with the embodied four components before assembly.

FIG. 2 depicts a partially assembled embodied urine control device with the device before positioning on a user, wherein the top cover is ready to mate with the fully assembled base portion once the base portion is applied to the user.

FIG. 3 depicts an unassembled embodied similar to that as shown in FIG. 1 but with an optional embodied strap system for securing the device to a user.

FIG. 4 depicts the front view of a fully assembled embodied urine control device.

FIG. 5 depicts the rear view of a fully assembled embodied urine control device

FIG. 6 depicts the bottom surface view of a fully assembled embodied urine control device.

### DESCRIPTION OF THE PREFERRED EMBODIMENTS

Before explaining the present invention in detail, it is important to understand that the invention is not limited in its application to the details of the construction illustrated and the steps described herein. The invention is capable of other embodiments and of being practiced or carried out in a variety of ways. It is to be understood that the phraseology and terminology employed herein is for the purpose of description and not of limitation.

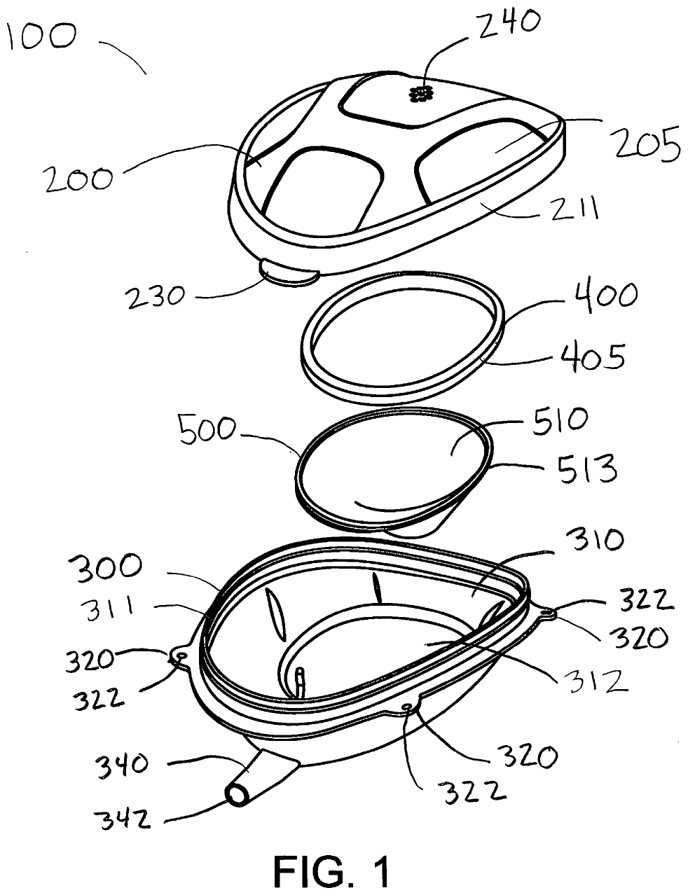

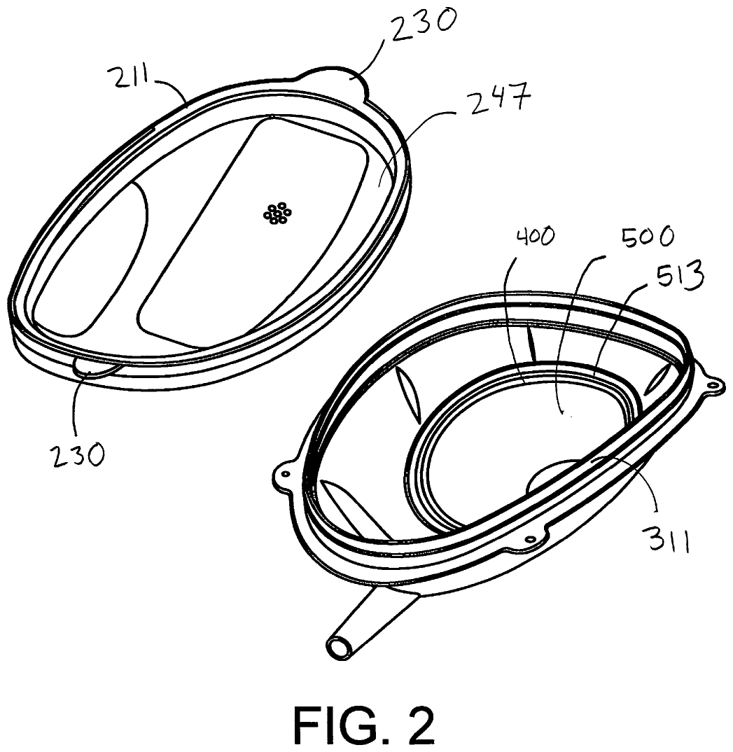

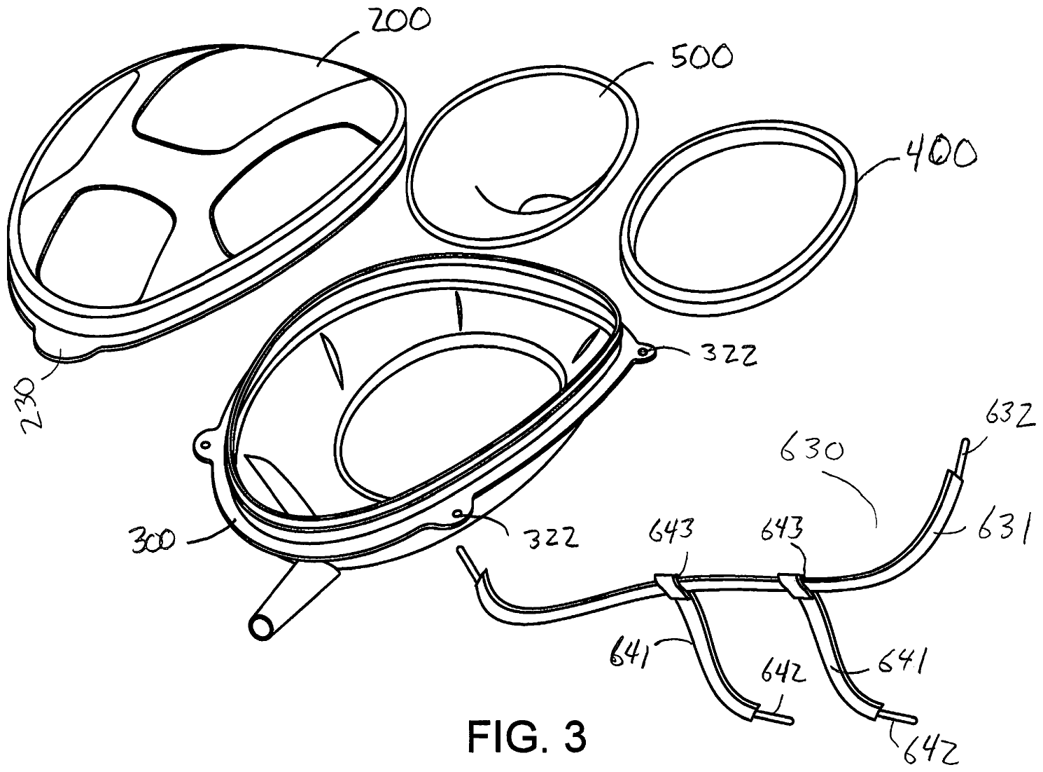

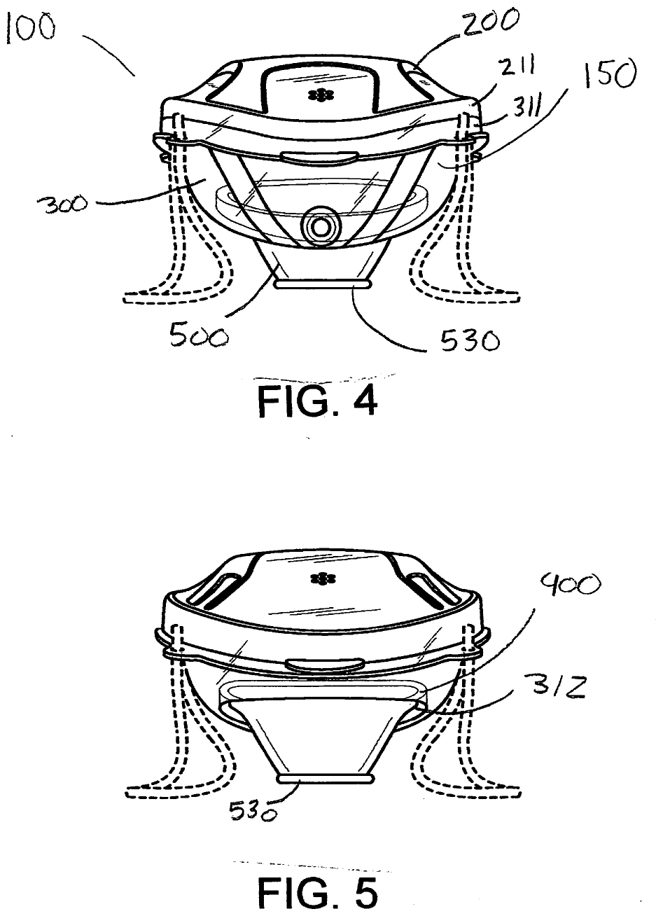

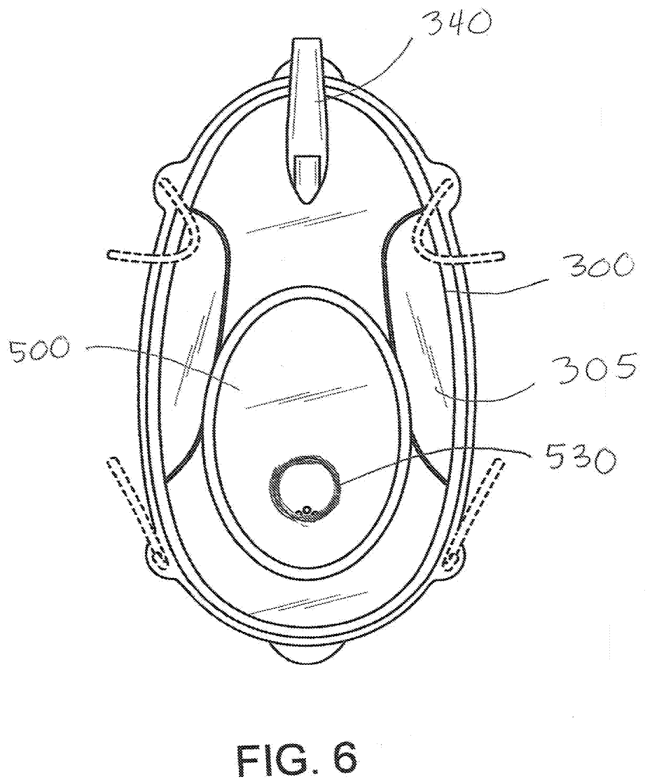

Referring now to the drawings, FIGS. 1-6, demonstrate one preferred embodiment of a urine control device as shown in its general environment. FIGS. 1 and 3 show a contemplated unassembled urine control device 100 comprising the four main components in FIG. 1 and a fifth optional user strap attachment component in FIG. 3. The four main components comprise of a top piece cover 200 which mates with a bottom base unit 300 and snap ring 400 which helps mechanically lock a membrane portion 500 onto the bottom base unit. Once the base assembly is completed as shown in FIG. 2 the base unit 300 mates with the top piece cover 100 to create a leak proof chamber 150 (shown in FIGS. 4-6).

The embodied top piece cover 200 shown throughout additionally has optional vent holes 240 when used in embodiments that use a pump system (not shown) to withdraw fluids from the chamber 150. The bottom base unit 300 has an internal cavity 310, which when defined by the internal surfaces of the top/cover piece 200 and bottom base unit 300 when mated together creates a leak proof chamber 150. The bottom base unit 300 bottom opening 312 to allow for a membrane portion 500 to be attached. The membrane portion 500 has a membrane opening so that at least the shaft of the penis and optionally the scrotum may be pulled through. The membrane opening is designed to stretch enough to expand to allow the genitalia to get through and then contract snugly against the genitalia to create a leak-proof seal. An additional cinch type mechanism such as a tie or elastic string may be optionally added to the membrane opening to assist with the leak-proof seal. Additionally, there is an attachment lip or ring around the top portion of the bottom piece used for mating with a paired attachment surface on the bottom part of the top piece. Further additional embodiments may include a strap support system comprising two or more strap supports. As shown in the current figures there are four strap supports with four strap support holes through which a strap end is pulled through in order to secure the device snugly to the patient.

When in use the healthcare personnel starts with the urine control device 100 in its two pieces the top and bottom portions and first places the bottom piece 300 onto the patient, by placing the bottom surface 305 of the bottom piece against the pelvic wall of the patient after pulling the patients genitalia through the membrane opening. In optional embodiments the personnel may pull only the shaft of the penis through and allow for the scrotum portion to be outside the chamber created by the device.

After the genitalia is secured within the internal cavity of the bottom piece the top piece cover is placed on the bottom piece and snap fit on creating a leak proof seal between the two pieces, and when the two pieces are mated together a cavity or chamber is created for the genitalia to reside in.

An additional embodiment includes a collection tube which draws the urine collected in the urine control device into a secondary collection reservoir so that urine is drawn away from the patient and the patients exposure to the urine on their genitalia surface is minimized. The collection tube may draw fluid out by mere capillary forces, gravitational pull or a form of negative pressure such as a vacuum, pressure bulb, or pump.

FIG. 2, demonstrates an embodied urine control device while assembled and including a strapping mechanism for further securing the device to the patient. The strapping shown secures around the legs and buttocks of the patient and is drawn tight against the patient by pulling the ends of the straps through strap support openings where they can be further tied or knotted to avoid loosening, or use an oxygen mask type one way draw which requires more force to release.

FIG. 3 shows another embodiment of the urine control device without the straps, but also wherein the top piece is either transparent or translucent so that the healthcare personnel can monitor the patients' irritation or effectiveness without removing the entire device.

FIG. 4 shows the bottom surface of the bottom piece which is the portion of the device which comes into closest contact with the patient. The bottom surface of the bottom piece may further include padding or diaper type material (not shown) which may create an additional leak barrier and also provide padding between the device and the patient. Additionally, FIG. 4 shows a clear view of the membrane and membrane opening relationship with the bottom piece and the optional strap supports, and collection tube. Additionally, shown is the optional collection tube support band which helps protect the collection tube from being pinched or kinked near the device but also may be modified to allow for a strapping anchor point.

Not shown in the figures is the proprietary nature of the materials used in the urine control device. The device may be made from any biocompatible materials, however optional embodiments use different materials with different properties throughout the device. In a first contemplated embodiment the bottom piece is made from a highly flexible material such as vinyl so that it can flex and conform while against the users body, it has properties very similar to an oxygen mask. The top piece contemplated is generally more rigid and inflexible and provides more of the structural rigidity necessary especially when using a vacuum pull to continually remove urine from the device. The membrane portion may be a thin fluid proof barrier layer with high elasticity such as found in condoms, or latex or nitrile gloves. Additionally, highly absorbent diaper like materials may be placed in the internal cavity for devices that do not rely on a collection tube, or in highly susceptible leak spots such as on the underside of the membrane. In a second contemplated embodiment the bottom piece is made of a more rigid material and a more flexible top cover piece is secured onto the bottom piece to create the chamber. When a more rigid bottom piece is used there may optionally be some padding absorbent material secured to the underside of the bottom piece and in contact with the patients skin.

What is claimed is:

1. A urine control device comprising of:
a top cover portion capable of mating to a base portion,
a base portion capable of mating with the top portion to create a leak proof chamber; wherein the base portion further comprises a membrane portion which is mechanically attached to the base portion via a snap ring;
further wherein the membrane assembly has a flexible opening which may secure to a user in a leak proof manner.
